(19) United States
(12) Patent Application Publication
(10) Pub. No.: US 2026/0108388 A1
(43) Pub. Date: Apr. 23, 2026

(54) SYSTEMS AND METHODS FOR DELIVERING ADJUSTABLE SHUNTING SYSTEMS

(71) Applicant: Shifamed Holdings, LLC, Campbell, CA (US)

(72) Inventors: Steven Pham, Campbell, CA (US); Gregory Andaloro, Santa Cruz, CA (US)

(21) Appl. No.: 19/120,463

(22) PCT Filed: Nov. 1, 2023
(86) PCT No.: PCT/US2023/078308

§ 371 Date: Apr. 11, 2025

Related U.S. Application Data
(60) Provisional application No. 63/421,941, filed on Nov. 2, 2022.
(60) Provisional application No. 63/578,953, filed on Aug. 25, 2023.

(51) Int. Cl. A61F 9/007; A61B 17/00; A61B 17/30

(52) U.S. Cl. CPC A61F 9/00781; A61B 2017/00367; A61B 17/30; A61B 2217/007

(57) ABSTRACT

The present technology is generally directed to shunt delivery systems and associated methods for delivering and deploying adjustable shunts. In some embodiments, the delivery systems include a first arm and a second arm configured to be coupled to and movable relative to one another, and a locking mechanism operably coupled to the first arm and configured to releasably engage the second arm to at least partially prevent movement of one or both of the first arm and the second arm relative to one another. During an adjustable shunt implantation procedure, a user can actuate the locking mechanism to at least partially prevent movement of one or both of the first arm and the second arm relative to one another, receive the adjustable shunt at the implant delivery system, and position the adjustable shunt at a target location within the patient.

### CROSS-REFERENCE TO RELATED APPLICATION(S)

This application claims priority to U.S. Provisional Patent Application No. 63/421,941 filed, Nov. 2, 2022, and U.S. Provisional Patent Application No. 63/578,953 , filed Aug. 25, 2023, both of which are hereby incorporated by reference in their entireties.

### TECHNICAL FIELD

The present technology generally relates to systems and methods for delivering implantable medical devices and, in particular, to delivery systems and associated methods for delivering intraocular shunts.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen). Conventional, early shunting systems (sometimes referred to as minimally invasive glaucoma surgery devices or “MIGS” devices) have shown clinical benefit; however, there is a need for improved shunting systems, systems for delivering such shunting systems, and techniques for addressing elevated intraocular pressure and risks associated with glaucoma. For example, there is a need for shunting systems capable of adjusting the therapy provided, including the flow rate between the two fluidly connected bodies. Further, there is a need for delivery systems for effectively and precisely delivering such shunting systems to target treatment locations within patients.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A is a perspective view of a delivery system configured for delivery of an adjustable shunting system in accordance with select embodiments of the present technology.

FIG. 1B an enlarged perspective view of shunt-receiving portions of the delivery system of FIG. 1A.

FIG. 1C is an enlarged perspective view of shunt-receiving features of FIG. 1B.

FIG. 1D is a bottom view of the delivery system of FIG. 1A and a sealing cap configured in accordance with embodiments of the present technology.

FIG. 2 is a perspective view of two tips of another intraocular shunt delivery system configured in accordance with embodiments of the present technology.

FIG. 3 is an enlarged perspective view of shunt-receiving features of another intraocular shunt delivery system configured in accordance with embodiments of the present technology

FIG. 4 is a perspective view of another intraocular shunt delivery system configured in accordance with embodiments of the present technology.

FIG. 5 is a perspective view of another intraocular shunt delivery system configured in accordance with embodiments of the present technology.

FIG. 6A is a perspective view of another intraocular shunt delivery system configured in accordance with embodiments of the present technology.

FIG. 6B is an enlarged side view of a locking mechanism of the intraocular shunt delivery system of FIG. 6A.

FIG. 6C is an enlarged side view of a locking mechanism of another intraocular shunt delivery system configured in accordance with embodiments of the present technology.

FIG. 7A is a perspective view of an intraocular shunt delivery system loading device configured in accordance with embodiments of the present technology.

FIG. 7B is a top view of the intraocular shunt delivery system loading device of FIG. 7A.

FIGS. 8A-8C illustrate various stages of an operation for deploying a shunting system into a human eye using the delivery system of FIGS. 1A and 1B, in accordance with select embodiments of the present technology.

### DETAILED DESCRIPTION

The present technology is generally directed to delivery systems and associated methods for delivering and deploying adjustable shunting systems. A delivery system configured in accordance with embodiments of the present technology can include a first arm, a second arm, and a locking mechanism. The first arm and/or the second arm can be configured to be coupled to and movable relative to one another. The locking mechanism can be operably coupled to the first arm and configured to releasably engage the second arm to at least partially prevent movement of one or both of the first arm and the second arm relative to one another. In some embodiments, the delivery system can include a stopping component configured to at least partially prevent movement of one or both of the first arm and the second arm toward one another. For example, the stopping component can be coupled to the second arm such that movement of one or both of the first arm and the second arm toward one another can cause the stopping component to contact the first arm and thereby prevent, or at least partially prevent, further movement of one or both of the first arm and the second arm toward one another. In these and/or other embodiments, the first arm can include a first tip and the second arm can include a second tip. The first tip and the second tip can be configured to releasably receive the adjustable shunting system when the locking mechanism releasably engages the second arm. In at least some embodiments, for example, one or both of the first tip and the second tip can include a slot or channel within which at least a portion of the adjustable shunting system can be positioned. During an adjustable shunting system implantation procedure, the adjustable shunting system can be positioned (e.g., held in position at least partially between the first tip and the second tip, and a user can actuate the locking mechanism to at least partially prevent movement of one or both of the first arm and the second arm relative to one another, position the adjustable shunting system at a target location within the patient, and deploy the adjustable shunting system at the target location.

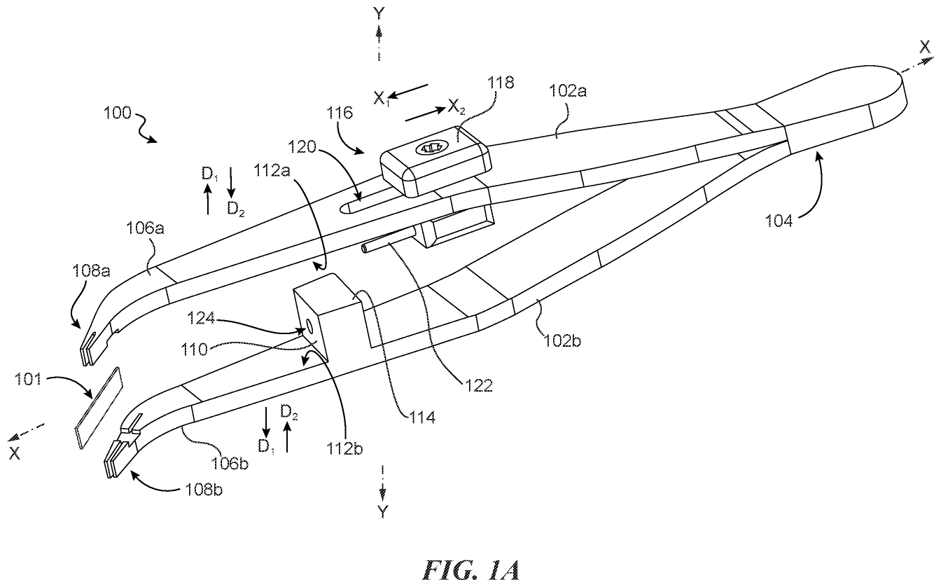

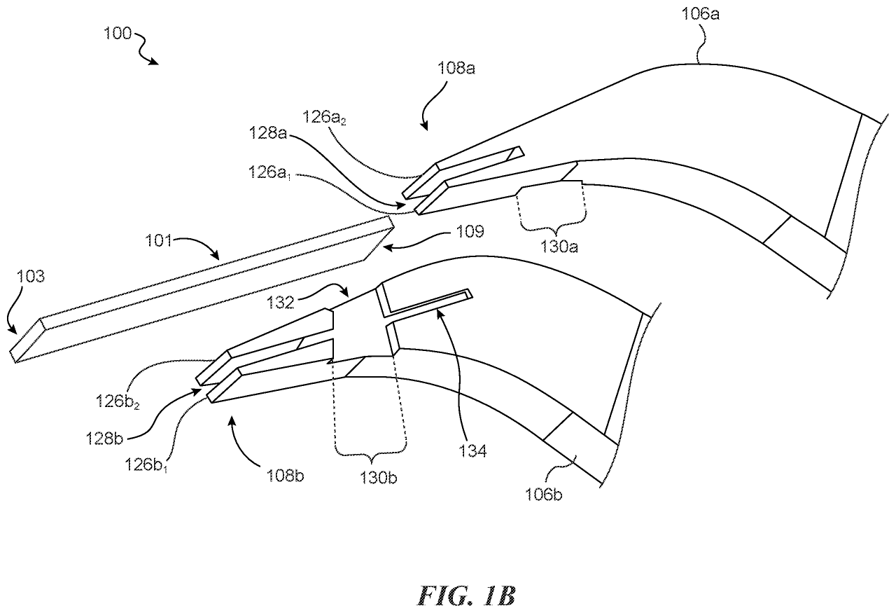

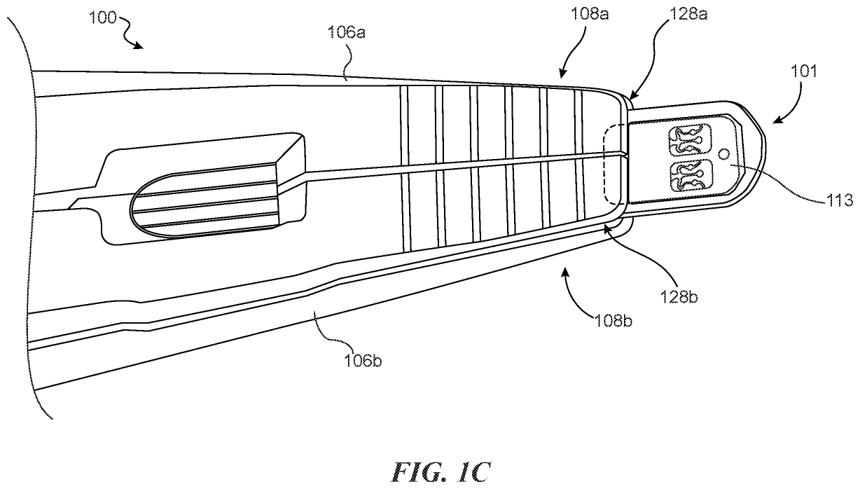

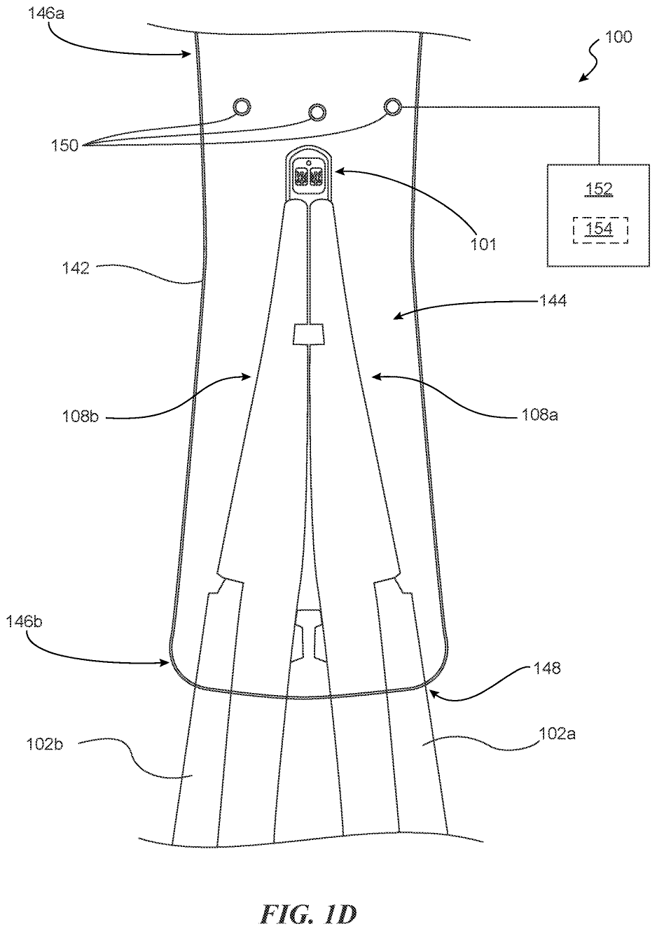

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims but are not described in detail with respect to FIGS. 1A-8C.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye (including the posterior chamber), or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIG. 1A is a perspective view of a delivery system 100 (“system 100”) configured for delivery of an adjustable shunting system 101 (shown schematically in FIG. 1A) in accordance with select embodiments of the present technology. The system 100 includes one or more biasing members or arms 102 (individually identified as a first arm 102a and a second arm 102b in FIG. 1A). At least a portion of the first arm 102a and the second arm 102b extend generally parallel to each other along a longitudinal axis X. Individual ones of the arms 102 can be formed using additive manufacturing, injection molding, and/or another suitable manufacturing process, e.g., using one or more metals (e.g., stainless steel, aluminum, etc.), polymers (e.g., Poly(methyl methacrylate), Polyethylene Terephthalate, etc.), and/or one or more other suitable materials. Each of the arms 102 can be configured to be movably coupled to one or more of the other arms 102. In the illustrated embodiment, for example, the first arm 102a is movably coupled to the second arm 102b at a proximal coupling portion or joint 104, such that one or both of the first and second arms 102a-b can be moved (e.g., deflected, pivoted, rotated, etc.) relative to one another, e.g., about the joint 104, in a first or outward direction D1 and/or a second or inward direction D2.

Each of the arms 102 can include one or more gripper or tip portions 106 (individually identified as a first tip 106a and a second tip 106b in FIG. 1A). Individual ones of the tips 106 can be formed using additive manufacturing, injection molding, and/or another suitable manufacturing process, e.g., using one or more metals (e.g., stainless steel, aluminum, etc.), polymers (e.g., Poly(methyl methacrylate), Polyethylene Terephthalate, etc.), and/or one or more other suitable materials. Although the system 100 includes a pair of arms 102a-b and a pair of tips 106a-b in the embodiment illustrated in FIG. 1A, in other embodiments the system 100 can include more tips 106 and/or arms 102. For example, the number of tips 106 can be less than, equal to, or greater than the number of arms 102.

Each of the tips 106 can include a distal or shunt-receiving feature 108 configured to receive the adjustable shunt 101. In the illustrated embodiment, for example, the first tip 106a includes a first shunt-receiving feature 108a configured to receive at least a first portion of the adjustable shunt 101 and the second tip 106b includes a second shunt-receiving feature 108b configured to receive at least a second portion of the adjustable shunt 101. The tips 106 can be angled relative to one or more other portions of the arms 102, and/or the longitudinal axis X. In the illustrated embodiment, for example, the shunt receiving portions 108 can be curved away from the longitudinal axis and/or toward a direction perpendicular to the directions D1, D2 in which the arms are configured to move, e.g., such that the curvature of the tips 106 mirror one another about the longitudinal axis X.

The arms 102 can be moved relative to each other and/or the joint 104, such as along a lateral or width-wise axis Y, to adjust or change a distance/spacing between the tips 106. In the illustrated embodiment, for example, individual ones of the arms 102 are configured to be biased in the first direction D1, axially outward along the lateral axis Y, and can be bent or deflected in the second direction D2 opposite the first direction DI, axially inward along the lateral axis Y. Movement of one or both of the arms 102 in the second direction D2 can secure or hold the adjustable shunt 101 at least partially between the tips 106, such as at least partially within one or both of the shunt-receiving features 108 of the tips 106, such that the system 100 can be used to transport the adjustable shunt 101 and/or position the adjustable shunt 101 at least partially within a body cavity or region of a patient.

The system 100 can further include a stopping component or spacer 110 positioned to at least partially prevent movement of the arms 102 in the second direction D2. The stopping component 110 can be coupled to an inner surface portion 112b of the second arm 102b and can extend (e.g., perpendicularly) from the inner surface portion 112b to define an end surface or stopping surface 114 positioned to contact an inner surface portion 112a of the first arm 102a. Accordingly, movement of one or both of the arms 102 in the second direction D2 can cause the stopping surface 114 to contact the inner surface portion 112a, and/or another portion of the system 100 opposite the stopping surface 114, and thereby at least partially prevent further inward movement of one or both of the arms 102 toward each other in the second direction D2. Accordingly, the stopping component 110 is expected to reduce or prevent compression and/or other force-related stresses that can be induced in all or part of the system 100 (e.g., the tips 106) when the arms 102 are moved toward each other in the second direction D2. This is expected to reduce wear and/or other stress-related fatigue experienced by the system 100 when in use.

In some embodiments, the system 100 can include a coupling or locking mechanism 116 configured to inhibit or prevent movement of the arms 102 and/or the tips 106 relative to one another, such as after one or both of the arms 102 have been moved in the second direction D2 toward one another and/or axially inward along the Y-axis. In the illustrated embodiment, the locking mechanism 116 includes an actuator 118 slidably disposed through a slot 120 formed in the first arm 102a. The slot 120 can extend in a direction parallel or substantially parallel to the longitudinal axis X and the actuator 118 can be moved along the slot 120, as shown by arrows X₁ and X₂, to selectively allow or inhibit/prevent movement of the arms 102 relative to one another and/or the longitudinal axis X.

The locking mechanism 116 can be configured to operably engage a portion of the stopping component 110, for example, to lock or hold the arms 102 in an inwardly-deflected state. In the illustrated embodiment, for example, the actuator 118 of the locking mechanism 116 includes a locking component or shaft 122 and the stopping component 110 includes an aperture or locking recess 124 configured to receive at least a portion of the shaft 122. Movement of one or both of the arms 102 in the second direction D2 can position/align the locking recess 124 to receive the shaft 122. With the locking recess 124 and the shaft 122 so positioned/aligned, the actuator 118 can be moved distally, such as in the direction indicated by the arrow X₁, to cause at least a portion of the shaft 122 to move toward and/or be positioned within the locking recess 124 and thereby hold the arms 102 in the inwardly deflected state and/or otherwise prevent movement of the arms 102 relative to one another. Additionally, or alternatively, the actuator 118 can be moved proximally, such as in the direction indicated by the arrow X₂, to cause at least a portion of the shaft 122 to move away from and/or be positioned outside of the locking recess 124 and thereby allow the arms 102 to be moved away from each other in the first direction D1. In these and other embodiments, one or both of the arms 102 can be configured (e.g., biased) so as to automatically move in the first direction DI in response to proximal movement of the actuator 118 in the second direction X₂.

FIG. 1B is an enlarged perspective view of the shunt-receiving features 108 of the system 100. Each of the shunt-receiving features 108 of the tips 106 can include one or more shunt-coupling tines or fingers 126 (individually identified as a first finger 126a₁ and a second finger 126a₂ of the first shunt-receiving feature 108b₁ and a first finger 126b₁ and a second finger 126b₂ of the second shunt-receiving feature 108b₂ in FIG. 1B). In the illustrated embodiment, the first and second fingers 126a₁₋₂ of the first shunt-receiving feature 108a are spaced apart from one another to define a first shunt-receiving slot or channel 128a therebetween that extends in a direction parallel or substantially parallel to a length of the first shunt-receiving feature 108a. Likewise, the first and second fingers 126b₁₋₂ of the second shunt-receiving feature 108b are spaced apart from one another to define a second shunt-receiving slot or channel 128b therebetween that extends in a direction parallel or substantially parallel to a length of the second shunt-receiving feature 108b. The first slot 128a and the second slot 128b can be spaced apart from one another, as shown in FIG. 1B, and/or can be moved toward one another in response to movement of one or more of the tips 106 in the second direction D2 (FIG. 1A).

All or a portion of the adjustable shunt 101 (shown schematically) can be positioned between one or more of the first fingers 126a₁, 126b₁ and/or one or more of the second fingers 126a₂, 126b₂. In some embodiments, a distal portion 103 of the adjustable shunt 101 can extend distally beyond the slots 128 and the fingers 126 when the adjustable shunt 101 is carried by the system 100, while a proximal portion 109 of the adjustable shunt 101 can be received within the slots 128 and/or between individual ones of the fingers 126. In some embodiments, friction between the adjustable shunt 101 and individual ones of the fingers 126 can inhibit or prevent movement (e.g., unintentional movement) of the adjustable shunt 101 relative to the fingers 126. Additionally, or alternatively, one or both of the first and second fingers 126a₁, 126b₁ of the first tip 106a and/or one or both of the first and second fingers 126b₁, 126b₂ of the second tip 106b can be biased toward one another and configured to pinch or otherwise apply a compressive force to the adjustable shunt 101 when the adjustable shunt 101 is positioned within one or both of the slots 128a-b.

In some embodiments, one or more of the shunt-receiving features 108 can include a thinned or narrowed portions 130 (individually identified as a first narrowed portion 130a of the first tip 106a and a second narrowed portion 130b of the second tip 106b in FIG. 1B) configured to increase the flexibility of the tips 106. In at least some embodiments, for example, the shunt-receiving portions 108a-b can be configured to bend or deflect at or near the corresponding narrowed portions 130a-b, such as in response to movement of one or both of the arms 102 toward one another. Additionally, or alternatively, the narrowed portions 130 can define a transverse slot or window 132 extending through the tip 106 in a direction perpendicular or generally perpendicular to a length of the tip 106. In such embodiments, at least a portion of the adjustable shunt 101 can be aligned with and/or accessible through the transverse slot 132, for example, so that a user can visualize whether the adjustable shunt is positioned. In some embodiments, one or both of the slots 128 can extend proximally to and/or beyond the transverse slot 132. In the illustrated embodiment, for example, the second slot 128b includes a proximal portion 134 extending proximally from the transverse slot 132. Although not visible in FIG. 1B, the first slot 128a can also include a proximal portion extending proximally from a corresponding transverse slot in the first tip 106a.

FIG. 1C is an enlarged perspective view of the shunt-receiving features 108 of the system 100. Other portions of the system 100 are omitted for the purposes of clarity. The shunt 101 can include a flow control assembly or plate 113 configured to control the flow of fluid within and/or through the shunt 101. When the shunt 101 is received within the slots 128a-b, the tips 106 can contact and/or capture at least a portion of the flow control assembly 113, e.g., as shown in dashed-line in FIG. 1C. Because the flow control assembly 113 is expected to be stiffer than one or more other (e.g., adjacent, surrounding, etc.) portions of the shunt 101, engaging the flow control assembly 113 with the tips 106 is expected to help stabilize the shunt 101 during insertion, e.g., to prevent, or at least partially prevent, bending and/or deflection of the shunt 101, including bending and/or deflection at or near the flow control assembly 113.

FIG. 1D is a bottom view of the system 100 and a sealing cap or shunt covering component 142 (“cap 142”) configured in accordance with embodiments of the present technology. The cap 142 can define an interior or chamber 144 configured to receive at least a portion of the system 100. In the illustrated embodiment, for example, at least part of the shunt-receiving portions 108a-b and the arms 102a-b are positioned within the chamber 144. Accordingly, in at least some embodiments, the cap 142 can be placed on and/or about the system 100 such that all or part of the adjustable shunt 101 is positioned within the chamber 144. The cap 142 includes a first or distal end portion 146a and a second or proximal end portion 146b opposite the distal end portion 146a. The proximal end portion 146b can be configured to form a substantially fluid-impermeable seal 148 with the system 100, including with one or more of the arms 102 and/or the shunt-receiving portions 108a-b. The distal end portion 146a can be open, closed off, and/or configured to be sealable. Accordingly, the chamber 144 and/or one or more objects (e.g., the adjustable shunt 101) positioned therein can be protected from the environment external to the cap 142. In some embodiments, positioning the adjustable shunt 101 within the chamber 144 is expected to reduce or prevent damage, contamination, and/or other interference that may inhibit or otherwise alter the operation of the adjustable shunt 101. For example, the cap 142 can be placed around the adjustable shunt 101, the chamber 144 can be sealed, sterilized, and/or sanitized, and then the system 100 and cap 142 can together be used to transport the adjustable shunt 101. The cap 142 can be removed prior to implantation of the adjustable shunt 101.

The cap 142 can include one or more apertures or ports 150 (which can also be referred to as “priming ports,” “priming inlets,” and/or the like), each of which can be fluidly coupled to the chamber 144 of the cap 142. Fluid (e.g., priming fluid) introduced through one or more of the ports 150 can enter the chamber 144 and flow into the adjustable shunt 101. The fluid can be drawn through one or more of the ports 150, such as in response to a reduced pressure or vacuum generated within the chamber 144, injected through one or more of the ports 150, such as using a syringe or other fluid delivery tool, and/or via one or more other suitable fluid delivery techniques. Because the cap 142 can be configured to form the substantially fluid-impermeable seal 148, all or substantially all fluid flow into and/or out of the chamber 144 can be through one or more of the ports 150 and/or one or both of the distal end portion 146a and the proximal end portion 146b.

The fluid introduced into the chamber 144 can be used to prime the adjustable shunt 101 before and/or during a procedure involving the system 100 and/or the adjustable shunt 101. In some embodiments, for example, the adjustable shunt 101 can be primed by a physician. In other embodiments, the adjustable shunt 101 can be primed during a manufacturing process for the system 100, such as prior to placement of the cap 142. Priming the adjustable shunt 101 can introduce fluid (e.g., priming fluid) into at least a portion of the adjustable shunt 101 to thereby reduce resistance to initiating fluid flow through the adjustable shunt 101, e.g., after the adjustable shunt 101 has been implanted. In the illustrated embodiment, for example, a fluid delivery tool 152 (shown schematically in FIG. 1D), such as a syringe, is loaded with priming fluid 154 and used to flow/inject the priming fluid 154 into the chamber 144 of the cap 142 via one or more of the ports 150. Once within the chamber 144, the priming fluid 154 can flow into the adjustable shunt 101. For example, the force/pressure generated by the fluid delivery tool 152 and/or with which the priming fluid 154 is injected into the chamber 144 can be greater than a fluid inflow resistance of the adjustable shunt 101, such that all, or at least a portion, of the priming fluid 154 is expected to flow into the adjustable shunt 101. Additionally, or alternatively, the priming fluid 154 injected into the cap 142 can build up within the chamber 144 can to create a pressure gradient between the chamber 144 and the interior of the adjustable shunt 101; the pressure gradient can increase, e.g., until it exceeds the fluid inflow resistance of the adjustable shunt 101 and causes all, or at least a portion, of the priming fluid 154 to flow into the adjustable shunt 101. In these and other embodiments, a reduced pressure or vacuum can be generated within the chamber 144 and used to draw the priming fluid 154 into the chamber 144 from the fluid delivery tool 152 or another fluid source.

In some embodiments, a reduced pressure or vacuum can be generated within the adjustable shunt 101 and used to drawing the priming fluid 154 into at least a portion of the adjustable shunt 101. For example, the chamber 144 and/or the adjustable shunt 101 can be evacuated via one or more of the ports 150 and/or one or both of the distal end portion 146a and the proximal end portion 146b. In some embodiments, the vacuum generated within the chamber 144 and/or the adjustable shunt 101 can be used to directly draw the priming fluid 154 into the chamber 144 and/or the adjustable shunt 101 (e.g., during and/or in concert with the evacuation of the chamber 144 and/or the adjustable shunt 101). In other embodiments, the chamber 144 and/or the adjustable shunt 101 can be sealed to store the vacuum such that, at some time after the vacuum is generated, the vacuum can be used to draw the priming fluid 154 into the chamber 144 and/or the adjustable shunt 101. In these and other embodiments, the vacuum generated within the chamber 144 and/or the adjustable shunt 101 can be used to draw fluid into the chamber 144 and/or the adjustable shunt 101 via one or more of the ports 150 and/or one or both of the distal end portion 146a and the proximal end portion 146b.

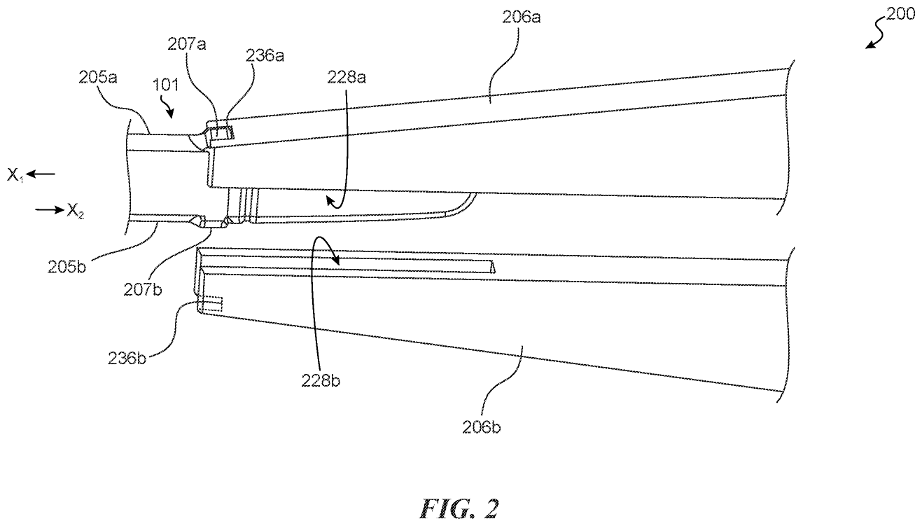

FIG. 2 is a perspective view of two tips 206a-b of another intraocular shunt delivery system 200 (“system 200”) configured in accordance with embodiments of the present technology. Other portions of the system 200 are omitted for the purposes of clarity. At least some aspects of the system 200 can be generally similar or identical in structure and/or function to the system 100 of FIGS. 1A and 1B. For example, the system 200 can be configured to carry the adjustable shunt 101 and/or used to implant the adjustable shunt 101 within a patient's eye.

The system 200 can include one or more tips 206 (individually identified as a first tip 206a and a second tip 206b in FIG. 2). The tips 206 can be standalone components or part of a larger assembly. In at least some embodiments, for example, individual ones of the tips 206 can be coupled to one or more of the arms 102 of the system 100 of FIGS. 1A and 1B, such as in lieu of the tips 106 thereof. Each of the tips 206 can include a respective first shunt-receiving recess or channel 228 (individually identified as a first recess 228a of the first tip 206a and a second recess 228b of the second tip 206b in FIG. 2) configured to receive at least a portion of the adjustable shunt 101. In the illustrated embodiment, for example, the recesses 228a, 228b are configured to receive opposite (e.g., left and right) sides 205a and 205b, respectively, of the adjustable shunt 101. In some embodiments, individual ones of the recesses 228 can include an indent or notch 236 (individually identified as a first notch 236a of the first recess 228a and a second notch 236b of the second recess 228b in FIG. 2) positioned to receive a corresponding projection or wing 207 (individually identified as a first wing 207a and a second wing 207b in FIG. 2) of the adjustable shunt 101. In the illustrated embodiment, for example, the first notch 236a is defined in a distal end of the first tip 206a and positioned to receive the first wing 207a and the second notch 236b is defined in a distal end of the second tip 206b and positioned to receive the second wing 207b.

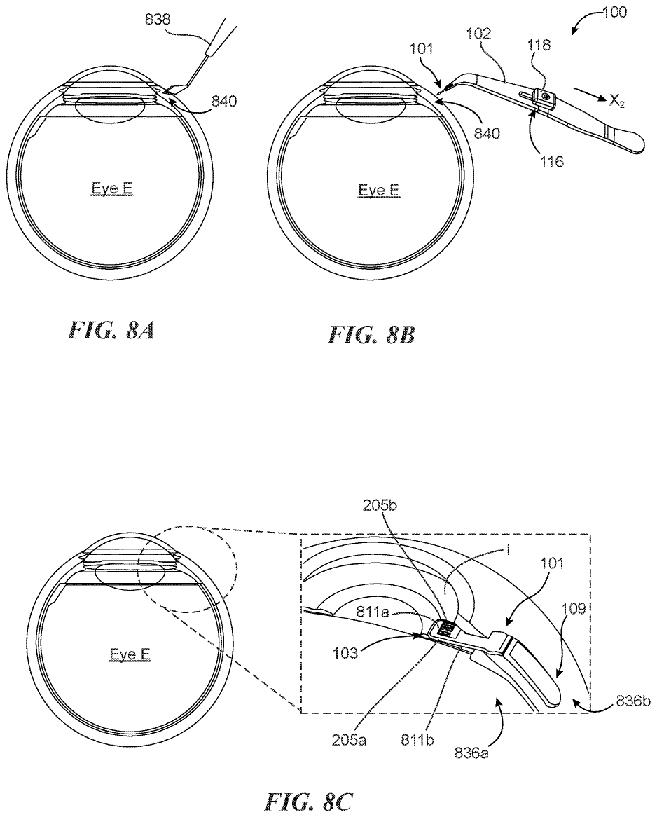

In operation, such as during an implantation procedure, the tips 206 can be pressed together to pinch or clamp the adjustable shunt 101 between the tips 206, e.g., to inhibit or even prevent movement of the adjustable shunt 101 relative to one or more of the tips 206. With the tips 206 clamping the adjustable shunt 101, the tips 206 can be used to position the adjustable shunt 101 at or near a target implant location, e.g., within a patient's eye, as described below with reference to FIGS. 8A-8C. In at least some embodiments, individual ones of the notches 236 can abut or contact the corresponding wing 207 of the adjustable shunt 101. In the illustrated embodiment, for example, the first notch 236a can contact the first wing 207a and the second notch 236b can contact the second wing 207b. Accordingly, the tips 206 can be used to push or press the adjustable shunt 101 in the first direction X₁ (e.g., distally), such as during an adjustable shunt implantation procedure, without or substantially without the adjustable shunt 101 sliding or moving in the second direction X₂ (e.g., proximally) relative to one or both of the tips 206.

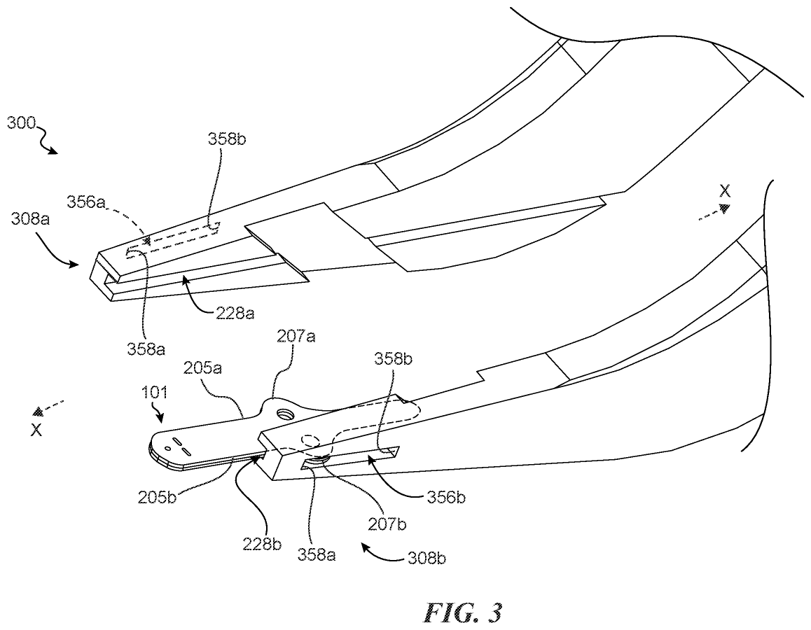

FIG. 3 is an enlarged perspective view of shunt-receiving features 308a-b of another intraocular shunt delivery system 300 (“system 300”) configured in accordance with embodiments of the present technology. Other portions of the system 300 are omitted for the purposes of clarity. At least some aspects of the system 300 can be generally similar or identical in structure and/or function to the system 100 of FIGS. 1A and 1B and/or the system 200 of FIG. 2. For example, the system 300 includes shunt-receiving features 308 (individually identified as a first shunt-receiving feature 308a and a second shunt-receiving feature 308b) that are configured to carry the adjustable shunt 101. The first shunt-receiving feature 308a includes the first recess 228a (FIG. 2) and the second shunt-receiving feature 308b includes the second recess 228b (FIG. 2). Additionally, the first shunt-receiving feature 308a includes a first slot 356a extending into and/or through the first recess 228a and the second shunt-receiving feature 308b includes a second slot 356b extending into and/or through the second recess 228b. The first slot 356a and the second slot 356b are referred to collectively as “the slots 356.”

Each of the slots 356 can be configured to receive a corresponding portion of the shunt 101. In the illustrated embodiment, for example, the first slot 356a is configured to receive the first wing 207a of the shunt 101 (e.g., at least when the first side 205a of the shunt 101 is positioned within the first recess 228a) and the second slot 356b is configured to receive the second wing 207b of the shunt 101 (e.g., at least when the second side 205b of the shunt 101 is positioned within the second recess 228b). Individual ones of the slots 356 can be defined at least partially by a first or distal surface 358a and a second or proximal surface 358b. The distal and proximal surfaces 358a-b can limit, or even prevent, movement of the shunt 101 relative to the shunt-receiving features 308. In the illustrated embodiment, for example, when the wings 207 are received in the corresponding slots 356, the wings 207 are spaced apart from but captured between the distal and proximal surfaces 358a-b, such that the distal surfaces 358a define a distal-most extent that the shunt 101 can move relative to the shunt-receiving features 308 and the proximal surfaces 358b define a proximal-most extent that the shunt 101 can move relative to the shunt-receiving features 308. In other embodiments, the distal surfaces 358a and the proximal surfaces 358b can contact the wings 207 when the wings 207 are received in the corresponding slots 356, e.g., to prevent any movement of the shunt 101 relative to the shunt-receiving features 308.

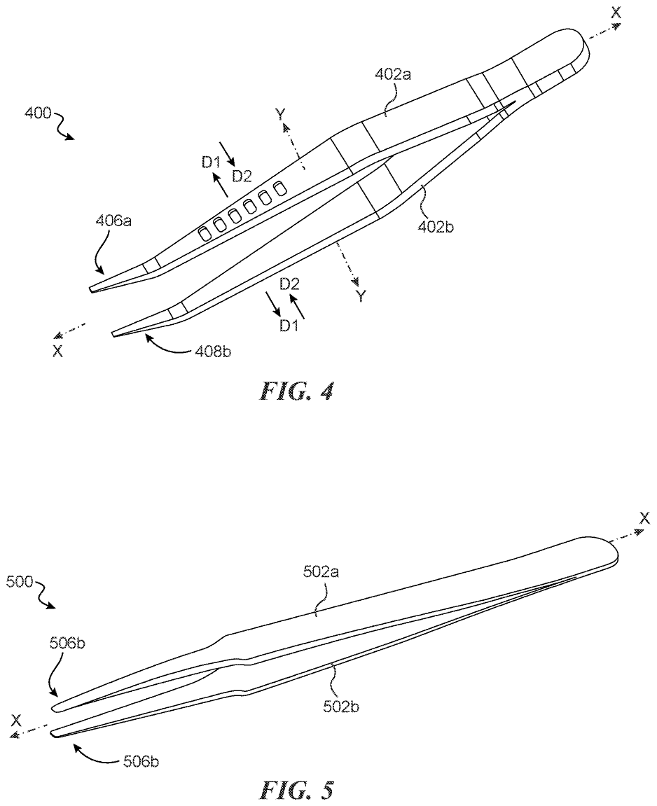

FIG. 4 is a perspective view of another intraocular shunt delivery system 400 (“system 400”) configured in accordance with embodiments of the present technology. At least some aspects of the system 400 can be generally similar or identical in structure and/or function to the system 100 of FIGS. 1A-1D, the system 200 of FIG. 2, and/or the system 300 of FIG. 3. For example, the system 400 includes a first arm 402a having a first tip portion 406a and a second arm 402b having a second tip portion 406b. However, the tip portions 406a-b have different curvature than the tips 106 of FIGS. 1A-1D. That is, rather than being curved to mirror one another about the longitudinal axis X), the tips 406a-b can be curved in a same plane as the directions D1, D2 in which the arms 402a-b are configured to move. In the illustrated embodiment, for example, one of the tips 406a-b (e.g., the second tip 406b) is curved toward the longitudinal axis X andor the other of the tips 406a-b (e.g., the first tip 406a), and the other of the tips 406a-b (e.g., the first tip 406a) is curved away from the longitudinal axis X. This curvature of the tips 406a-b is expected to provide better anatomical access, e.g., to a body region within a patient's eye within which an adjustable shunt is to be implanted, and/or is expected to be more intuitive for a user to manipulate during an implantation procedure. In other embodiments, the tips 406a-b, and/or any of the other tips (e.g., the tips 106 of FIGS. 1A-1D) described herein, can have other curvature based, at least in part, on anatomical access, user preference, intuitiveness, etc. One example of alternative curvature is shown in FIG. 5.

FIG. 5, for example, is a perspective view of another intraocular shunt delivery system 500 (“system 500”) configured in accordance with embodiments of the present technology. At least some aspects of the system 500 can be generally similar or identical in structure and/or function to the system 100 of FIGS. 1A-1D, the system 200 of FIG. 2, the system 300 of FIG. 3, and/or the system 400 of FIG. 4. For example, the system 500 includes a first arm 502a having a first tip portion 506a and a second arm 502b having a second tip portion 506b. However, compared to the tips portion 106a-b and 406a-b of FIGS. 1A-1D and FIG. 4, respectively, the tip portions 506a-b are not curved relative to other portions of the arms 502a-b.

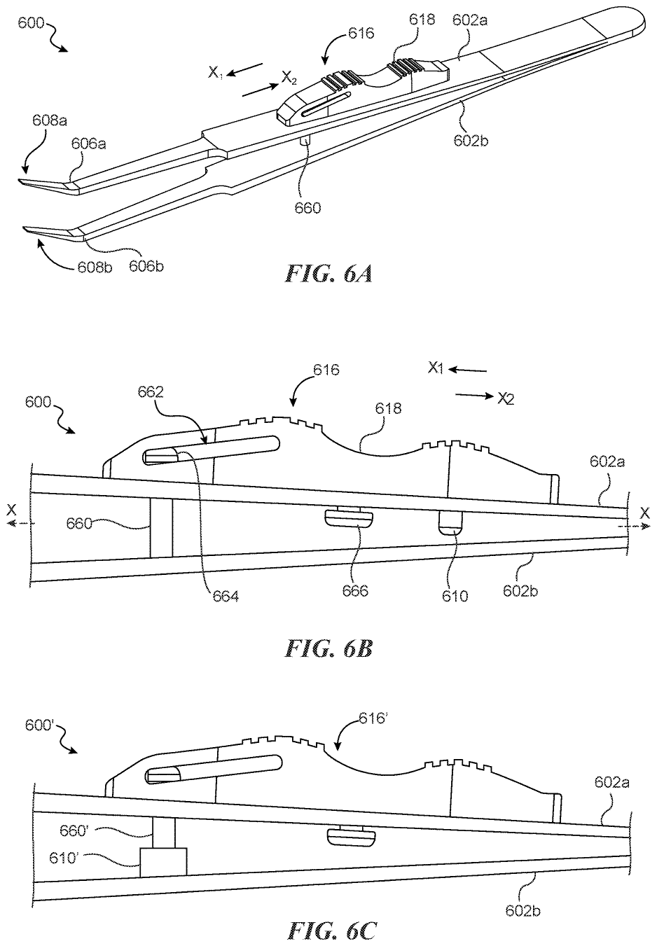

FIG. 6A is a perspective view of another intraocular shunt delivery system 600 (“system 600”) configured in accordance with embodiments of the present technology. At least some aspects of the system 600 can be generally similar or identical in structure and/or function to the system 100 of FIGS. 1A-1D, the system 200 of FIG. 2, the system 300 of FIG. 3, the system 400 of FIG. 4, and/or the system 500. For example, the system 600 includes a first arm 602a having a first tip portion 606a and a second arm 602b having a second tip portion 606b. The tips portions 606a-b have the curvature described previously with reference to FIG. 4.

Additionally, the system 600 includes a locking mechanism 616. The locking mechanism 616 includes an actuator 618 and a drive shaft 660. In the illustrated embodiment, the actuator 618 is movably coupled to the first arm 602a and the drive shaft 660 is coupled to the second arm 602b. In other embodiments, the actuator 618 can be slidably coupled to the second arm 602b and the drive shaft 660 can be coupled to the first arm 602a. In these and/or other embodiments, at least a portion of the drive shaft 660 can be received within the actuator 618. Accordingly, during a procedure and/or at one or more other times, a user can move the actuator 618 relative to the first arm 602a, e.g., in the first direction X₁ or the second direction X₂, to change a spacing between the first arm 602a and the second arm 602b. For example, the user can move the actuator 618 in the first direction X₁ (e.g., distally) to decrease the spacing between the arms 602a-b, e.g., to grip an adjustable shunt between the tip portions 606, and/or the user can move the actuator 618 in the second direction X₂ (e.g., proximally) to increase the spacing between the arms 602a-b, e.g., to release the adjustable shunt.

FIG. 6B is an enlarged side view of the locking mechanism 616 of the system 600. Other portions of the system 600 are omitted for the purposes of clarity. As best shown in FIG. 6B, the actuator 618 can define an actuation channel 662, the drive shaft 660 can have a head or end portion 664 configured to be positioned within the actuation channel 662. In at least some embodiments, the head 664 of the drive shaft 660 is captured within the actuation channel 662. The actuation channel 662 can be angled relative to, e.g., the first arm 602a and, accordingly, moving the actuator 618 relative to the first arm 602a can slide or translate the head 664 through the actuation channel 662 and thereby change (e.g., increase or decrease) the spacing between the head 664 and the first arm 602a. Because the drive shaft 660 is coupled to the second arm 602b, changing the spacing between the head 664 and the first arm 602a can draw the second arm 602b toward the first arm 602a and/or drive the first arm 602 toward the second arm 602b. In the illustrated embodiment, the actuation channel 662 is angled radially outwardly from the longitudinal axis X in a distal-to-proximal direction (e.g., the second direction X₂), such that a distal end of the actuation channel 662 is closer to the first arm 602a than a proximal end. Accordingly, moving the actuator 618 in the first direction X₁ causes the head 664 to slide through the actuation channel 662, away from the first arm 602a, thereby increasing the spacing between the head 664 and the first arm 602a. Similarly, moving the actuator 618 in the second direction X₂ causes the head 664 to slide through the actuation channel 662, toward from the first arm 602a, thereby increasing the spacing between the head 664 and the first arm 602a. In at least some embodiments, the actuation channel 662 can be configured to hold or lock the head 664 within the actuation channel 662, e.g., in a proximal-most position, a distal-most position, and/or one or more other positions therebetween to prevent, or at least partially prevent, further changes to the spacing between the arms 602a-b unless/until the hold on the head 664 is released and/or the head 664 is otherwise allowed to move through the actuation channel 662. In the illustrated embodiment, the actuator 618 is movably coupled to the first arm 602a by a fastener 666, such as a screw, a pin, and/or a rivet. In other embodiments, the actuator 618 can be movably coupled to the arm 602a by one or more other fasteners and/or fastening techniques.

The system 600 can further include a stopping component or spacer 610 that can be at least generally similar or identical in structure and/or function to the stopping component 110 of FIG. 1A. In addition to reducing wear and/or other stress-related fatigue experienced by the system 600, as described previously with reference to FIG. 1A, the spacer 610 may also be configured to control an amount/magnitude of force (e.g., compressive force) applied to the adjustable shunt 101, e.g., by the tips 606a-b. For example, the spacer 610 can be sized so that, when the spacer 610 contacts the opposing arm 602, a distance between the tips 606a-b is equal to, or at least generally equal to, a thickness or height of the adjustable shunt 101. Accordingly, when a user presses the arms 602a-b together to grip the adjustable shunt 101 between the tips 606a-b, the spacer 610 can prevent, or at least partially prevent, the user from over-compressing, crushing, squishing, and/or otherwise damaging the adjustable shunt 110. In at least some embodiments, the arms 602a-b of the system can be “fully closed” when the spacer 610 contacts the opposing arm (e.g., the first arm 602a) of the system 600, e.g., to prevent further inward movement of the arms 602a-b.

FIG. 6C is an enlarged side view of a locking mechanism 616′ of another intraocular shunt delivery system 600′ (“system 600′”) configured in accordance with embodiments of the present technology. The system 600′ can be at least generally similar or identical in structure and/or function to the system 600. For example, the system 600′ includes a drive shaft 660′ at least generally similar or identical to the drive shaft 660 of the system 600, and a spacer 610′ at least generally similar or identical to the spacer 610 of the system 600. However, the spacer 610′ is coupled to the second arm 602b and the drive shaft 660′ of the system 600′ is coupled to the second arm 602b through the spacer 610′. In some embodiments, the space 610′ is slidably positioned around the drive shaft 660′. In other embodiments, the spacer 610′ can be a widened portion of the drive shaft 660′.

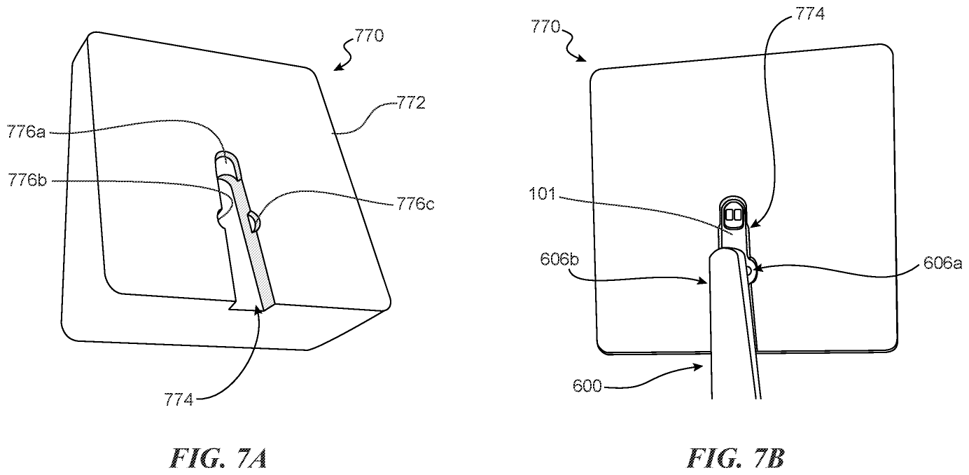

FIG. 7A is a perspective view of an intraocular shunt delivery system loading device 770 (“loading device 770”) configured in accordance with embodiments of the present technology. The loading device 770 includes a body 772 that defines a loading channel 774. The loading channel 774 can include one or more contact portions 776 (individually identified as a first contact portion 776a, a second contact portion 776b, and a third contact portion 776c). Each of the contact portions 776 can be configured to receive a corresponding portion of the adjustable shunt 101 (e.g., FIG. 3). In the illustrated embodiment, for example, the first contact portion 776a is configured to receive the distal end portion 103 (FIG. 1B) of the adjustable shunt 101, the second contact portion 776b is configured to receive one of the wings 207a-b (FIG. 3) of the adjustable shunt 101, and the third contact portion 776c is configured to receive the other of the wings 207a-b. In these and/or other embodiments, one or more of the contact portions 776 can be configured to contact other portions of the adjustable shunt 101, the loading channel 774 can include more contact portions configured to contact these and/or other portions of the adjustable shunt 101, and/or the loading channel 774 can include a lesser number of contact portions 776.

FIG. 7B is a top view of the loading device 770. Referring to FIGS. 7A and 7B together, the loading channel 774 can be configured to allow an intraocular shunt delivery system, such as the system 100 of FIGS. 1A-1D, the system 200 of FIG. 2, the system 300 of FIG. 3, the system 400 of FIG. 4, the system 500 of FIG. 5, and/or the system 600 of FIGS. 6A and 6B, to grip or otherwise engage opposing sides of the adjustable shunt 101. For example, as shown in FIG. 7B, the adjustable shunt 101 is positioned within the loading channel 774 and the tips 606a-b are engaging opposing (e.g., top and bottom) sides of the adjustable shunt 101. Once the adjustable shunt 101 is secured between the tips 606a-b, the adjustable shunt 101 can be lifted from the loading channel 774, e.g., and positioned within a patient's eye. Although described with reference to the tips 606 of the system 600, the loading device 770 can be configured to be compatible with the tips and/or one or more other portions of any of the other systems described herein with reference to FIGS. 1A-6B.

FIGS. 8A-8C illustrate various stages of a procedure for deploying an adjustable shunt into a patient's eye E using the system 100 in accordance with embodiments of the present technology. In a particular example, the system 100 can be used to deploy the adjustable shunt 101 such that, after implantation, the adjustable shunt 101 is positioned to route fluid from an anterior chamber of the patient's eye E to a suitable outflow location, such as a subconjunctival bleb space (e.g., to treat glaucoma). In other embodiments, the system 200 of FIG. 2, the system 300 of FIG. 3, the system 400 of FIG. 4, the system 500 of FIG. 5, the system 600 of FIGS. 6A and 6B, and/or one or more other suitable delivery systems can be used to perform one or more of the stages illustrated in FIGS. 8A-8C.

Referring first to FIG. 8A, one or more tools 838 (e.g., a keratome) can be used to make one or more incisions 840 in the eye E. Referring next to FIG. 8B, the system 100 can receive the adjustable shunt 101 and can be used to insert the adjustable shunt 101 into the eye E through the incision 840. The locking mechanism 116 can be actuated to release the adjustable shunt 101 from the system 100. For example, the actuator 118 can be moved in the second direction X₂ to allow the arms 102 to move relative to one another and release the adjustable shunt 101, as described previously regarding FIGS. 1A and 1B.

As shown in FIG. 8C, the adjustable shunt 101 can be positioned in the eye E such that the first portion 103 of the adjustable shunt 101 is positioned in a first body region 836a (e.g., within the eye E) and the second portion 109 of the adjustable shunt 101 is positioned in a second body region 836b (e.g., exterior to the eye E). Accordingly, after implantation, fluid within the first body region 836a can flow/drain through the adjustable shunt 101 toward and/or into the second body region 836b. In some embodiments, the adjustable shunt 101 can be configured to assume a curved, bent, or preformed shape during/after implantation in the patient, as shown in FIG. 8C. Additionally, or alternatively, the system 100 can be configured to adjust the shape of the system 100 in situ, and/or one or more of the tools used for the procedure can be used to adjust the shape of the system 100 in situ.

Although in FIG. 8C the first portion 103 of the adjustable shunt 101 is illustrated as being positioned anterior to (e.g., in front of/above) the patient's iris I, in other embodiments the first portion 103 can be positioned posterior to (e.g., behind/below) the iris I (also referred to as “sub-iris” positioning). The sub-iris positioning of the adjustable shunt 101 is expected to reduce or prevent corneal endothelial disease and/or failure, for example, by reducing or preventing disruption to nutrient and/or other fluid and/or chemical transport to and/or through the corneal endothelium. In the illustrated embodiment, the adjustable shunt 101 is configured to receive fluid (e.g., aqueous) through one or more openings or inlets in an upper surface 811a of the adjustable shunt 101. In these and other embodiments, including when the adjustable shunt 101 has a sub-iris position, the adjustable shunt 101 can be configured to receive fluid through one or more lateral openings, e.g., positioned in one or more sides 205a, 205b of the adjustable shunt 101, and/or through one or more openings in a bottom surface 811b of the adjustable shunt 101.

In general, the adjustable shunt 101 can be configured to actuate and/or change its resistance to fluid flow in response to energy (e.g., laser energy) delivered from a source external to the patient. An example of such a configuration is described in detail in U.S. Pat. No. 11,291,585, and incorporated herein by reference for all purposes. In embodiments in which the adjustable shunt 101 is positioned sub-iris, the adjustable shunt 101 can be actuated using a number of techniques. For example, a portion of the iris I can be removed (e.g., iridectomy) to provide line-of-sight access to the adjustable shunt 101. Additionally, or alternatively, the adjustable shunt 101 can be positioned such that at least a portion of the adjustable shunt 101 (e.g., at least part of the first portion 103) can be exposed when the eye E undergoes pupil dilation. In such embodiments, the pupil of the eye E can be dilated and then energy can be delivered to actuate the adjustable shunt 101. In these and other embodiments, the energy can be targeted using a first energy source (e.g., a first laser) configured to transmit first energy (e.g., targeting energy) at or near a first wavelength to which the iris I is translucent or transparent, and then a same or different energy source can transmit second energy (e.g., actuating energy) at a second wavelength different than the first wavelength.

In some embodiments, the adjustable shunt 101 can be configured to reduce or prevent cellular growth onto, over, and/or around at least a portion of the adjustable shunt 101. In at least some embodiments, for example, all or a portion of the adjustable shunt 101 can include one or more radioisotopes configured to inhibit or prevent cellular growth. The radioisotopes can include Phosporous-32, Strontium-89, Strontium-90, Yttrium-90, and/or another suitable radioisotope. Individual ones of the radioisotopes can emit alpha, beta, and/or gamma radiation, each of which are expected to inhibit or prevent cellular growth on, over, and/or near the adjustable shunt 101. For a given patient, the radioisotope(s) used with the adjustable shunt 101 can be selected based at least partially on the half-life of the radioisotope, the type of radiation, and/or the energy of the emitted alpha, beta, and/or gamma particles. The radioisotopes can be naturally-occurring or manufactured (e.g., using a cyclotron, reactor-produced, etc.).

### EXAMPLES

Several aspects of the present technology are described with reference to the following examples:
1. A delivery system for an adjustable shunt for treating a patient, the delivery system comprising:
   a first arm including a first tip portion;
   a second arm including a second tip portion, wherein the second arm is configured to be movably coupled to the first arm; and
   a locking mechanism operably coupled to the first arm and the second arm and configured to move the first arm and/or the second arm relative to one another in a movement plane,
   wherein the first tip portion is curved within the movement plane relative to one or more other portions of the first arm, and
   wherein the second tip portion is curved within the movement plane relative to one or more other portions of the second arm.
2. The delivery system of example 1 wherein the first tip portion is curved away from the second tip portion, and wherein the second tip portion is curved toward the first tip portion.
3. The delivery system of example 1 or 2 wherein the first tip portion and the second tip portion include one or more receiving features configured to carry an adjustable shunting system at least partially between the first tip portion and the second tip portion.
4. The delivery system of any of examples 1-3, further comprising:
   a drive shaft coupled to the second arm, wherein the drive shaft includes a head; and
   an actuator movably coupled to the first arm, wherein the actuator defines an actuation channel configured to capture the head of the drive shaft,
   wherein movement of the actuator relative to the first arm translates the head of the drive shaft through the actuation channel to move the first arm and/or the second arm relative to one another.
5. The delivery system of example 4 wherein the actuation channel is angled relative to the first arm.
6. The delivery system of example 5 wherein the first arm includes a proximal end portion and a distal end portion, and wherein the actuation channel is angled outwardly from the first arm in a proximal-to-distal direction.
7. A method for delivering an adjustable shunt to a patient via an implant delivery system, the method comprising:
   providing an implant delivery system including a first arm and a second arm movably coupled to the first arm, wherein the first arm has a first tip and the second arm has a second tip, and wherein an implantable adjustable shunt is positioned at partially between the first tip and the second tip; and
   positioning the adjustable shunt at a target location within an eye of the patient via the implant delivery system.
8. The method of example 7 wherein the first tip includes a first pair of fingers defining a first shunt-receiving slot therebetween, and wherein the second tip includes a second pair of fingers defining a second shunt-receiving slot therebetween, the method further comprising receiving the adjustable shunt in one or both of the first shunt-receiving slot and the second shunt-receiving slot.
9. The method of example 7 or 8 wherein the first tip includes a first shunt-receiving channel and the second tip includes a second shunt-receiving channel, the method further comprising receiving the adjustable shunt within one or both of the first shunt-receiving channel and the second shunt-receiving channel.
10. The method of example 9 wherein receiving the adjustable shunt within one or both of the first shunt-receiving channel and the second shunt-receiving channel includes receiving a proximal portion of the adjustable shunt between the first tip and the second tip such that a distal portion of the adjustable shunt extends distally beyond one or both of the first shunt-receiving channel and the second shunt-receiving channel.
11. The method of any of examples 7-10 wherein positioning the adjustable shunt at the target location further comprises actuating a locking mechanism of the adjustable shunt to allow movement of one or both of the first arm and the second arm relative to one another during operation.
12. The method of example 11 wherein actuating the locking mechanism includes moving an actuator of the locking mechanism away from the adjustable shunt.
13. The method of any of examples 7-12 wherein positioning the adjustable shunt at the target location includes positioning at least a portion of the adjustable shunt posterior to an iris of an eye of the patient.
14. The method of any of examples 7-13 wherein positioning the adjustable shunt at the target location includes positioning at least a portion of the adjustable shunt anterior to an iris of an eye of the patient.
15. The method of any of examples 7-14 wherein positioning the adjustable shunt at the target location includes positioning a first portion of the adjustable shunt in a first body region of the patient and positioning a second portion of the adjustable shunt in a second body region of the patient.
16. The method of any of examples 7-15, further comprising priming the adjustable shunt with a priming fluid before positioning the adjustable shunt within the patient.
17. The method of example 16 wherein priming the adjustable shunt includes causing the priming fluid to enter at least a portion of the adjustable shunt.
18. The method of example 16 or 17 wherein the implant delivery system includes a cap defining a chamber configured to contain the adjustable shunt, and wherein priming the adjustable shunt includes injecting the priming fluid into the chamber.
19. The method of any of examples 16-18 wherein the implant delivery system includes a cap defining a chamber configured to contain the adjustable shunt, and wherein priming the adjustable shunt includes:
   generating a vacuum within the chamber, and
   drawing, by the vacuum, the priming fluid into the chamber.
20. The method of example 18 or 19, further comprising removing the cap prior to positioning the adjustable shunt at the target location.
21. The method of any of examples 16-20 wherein priming the adjustable shunt includes providing the priming fluid to the chamber through one or both of a first end portion of the adjustable shunt and a second end portion of the adjustable shunt.
22. A delivery system for an adjustable shunt for treating a patient, the delivery system comprising:
   a first arm including a first tip;
   a second arm including a second tip, wherein the second arm is configured to be movably coupled to the first arm; and
   a locking mechanism operably coupled to the first arm and configured to releasably engage the second arm to at least partially prevent movement of one or both of the first arm and the second arm relative to one another,
   wherein
      the first tip and the second tip are each configured to releasably receive the adjustable shunt when the locking mechanism releasably engages the second arm, and
      one or both of the first tip and the second tip include a pair of fingers defining a channel therebetween, wherein the channel is configured to receive at least a portion of the adjustable shunt.
23. The delivery system of example 22 wherein one or both of the pair of fingers are biased toward one another to at least partially prevent movement of the adjustable shunt relative to the channel.
24. The delivery system of example 22 or 23 wherein the channel defines a notch configured to contact a corresponding wing of the adjustable shunt to at least partially prevent movement of the adjustable shunt relative to the channel.
25. The delivery system of example 24 wherein the channel is configured to receive a first portion of the adjustable shunt, wherein a second portion of the adjustable shunt is configured to extend distally beyond the channel, wherein the channel defines a notch configured to at least partially prevent proximal movement of the adjustable shunt relative to the channel.
26. The delivery system of any of examples 22-25 wherein one or both of the first tip and the second tip include a transverse slot extending through the associated first tip or the second tip and through which at least a portion of the adjustable shunt is visible at least when the adjustable shunt is received at the delivery system.
27. The delivery system of any of examples 22-26 wherein the first arm and/or the second arm are configured to be biased in a first direction outwardly from one another, and further wherein the first arm and/or the second arm are configured to be movable in a second direction opposite the first direction and inwardly toward one another.
28. The delivery system of any of examples 22-27 wherein the first arm includes a slot, and wherein the locking mechanism includes an actuator slidably disposed within the slot.
29. The delivery system of example 28 wherein the actuation mechanism is configured such that (i) movement of the actuator in a first direction causes the actuation mechanism to at least partially prevent movement of the first arm and the second arm relative to one another, and (ii) movement of the actuator in a second direction opposite the first direction causes the actuation mechanism to allow movement of one or both of the first arm and the second arm relative to one another.
30. The delivery system of any of examples 22-29, further comprising a stopping component positioned between the first arm and the second arm, wherein the stopping component is configured to at least partially prevent movement of one or both of the first arm and the second arm toward one another.
31. The delivery system of example 30 wherein the stopping component extends perpendicularly from a second inner surface portion of the second arm toward a first inner surface portion of the first arm.
32. The delivery system of example 30 or 31 wherein the stopping component includes a stopping surface configured to contact the first inner surface portion in response to movement of one or both the first arm and the second arm toward one another to at least partially prevent further movement of one or both of the first arm and the second arm toward one another.
33. The delivery system of any of examples 30-32 wherein the locking mechanism is configured to releasably engage the stopping component to at least partially prevent movement of one or both of the first arm and the second arm toward one another.
34. The delivery system of examples 30-32 wherein the stopping component includes a recess, and wherein the locking mechanism includes a shaft configured to be positionable within the recess to at least partially prevent movement of one or both of the first arm and the second arm relative to one another.
35. The delivery system of any of examples 22-34, further comprising a cap that defines a chamber configured to receive at least a portion of the first tip, the second tip, and the adjustable shunt therein.
36. The delivery system of example 35 wherein the cap defines an inlet, and wherein the chamber is configured to receive fluid via the inlet.
37. The delivery system of example 35 or 36 wherein the chamber is configured to contain a vacuum generated therein and receive priming fluid drawn from a priming fluid source via the vacuum.
38. The delivery system of any of examples 35-37 wherein the cap is configured to form a fluid-impermeable seal with at least one of the first arm, the first tip, the second arm, and the second tip.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the delivery systems for adjustable shunts described herein may be combined with any of the features of the other delivery systems described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A delivery system for an adjustable shunt for treating a patient, the delivery system comprising:
a first arm including a first tip portion;
a second arm including a second tip portion, wherein the second arm is configured to be movably coupled to the first arm; and
a locking mechanism operably coupled to the first arm and the second arm and configured to move the first arm and/or the second arm relative to one another in a movement plane,
wherein the first tip portion is curved within the movement plane relative to one or more other portions of the first arm, and
wherein the second tip portion is curved within the movement plane relative to one or more other portions of the second arm.

2. The delivery system of claim 1 wherein the first tip portion is curved away from the second tip portion, and wherein the second tip portion is curved toward the first tip portion.

3. The delivery system of claim 1 wherein the first tip portion and the second tip portion include one or more receiving features configured to carry an adjustable shunting system at least partially between the first tip portion and the second tip portion.

4. The delivery system of claim 1, further comprising:
a drive shaft coupled to the second arm, wherein the drive shaft includes a head; and
an actuator movably coupled to the first arm, wherein the actuator defines an actuation channel configured to capture the head of the drive shaft,
wherein movement of the actuator relative to the first arm translates the head of the drive shaft through the actuation channel to move the first arm and/or the second arm relative to one another.

5. The delivery system of claim 4 wherein the actuation channel is angled relative to the first arm.

6. The delivery system of claim 5 wherein the first arm includes a proximal end portion and a distal end portion, and wherein the actuation channel is angled outwardly from the first arm in a proximal-to-distal direction.

7. A method for delivering an adjustable shunt to a patient via an implant delivery system, the method comprising:
providing an implant delivery system including a first arm and a second arm movably coupled to the first arm, wherein the first arm has a first tip and the second arm has a second tip, and wherein an implantable adjustable shunt is positioned at partially between the first tip and the second tip; and
positioning the adjustable shunt at a target location within an eye of the patient via the implant delivery system.

8. The method of claim 7 wherein the first tip includes a first pair of fingers defining a first shunt-receiving slot therebetween, and wherein the second tip includes a second pair of fingers defining a second shunt-receiving slot therebetween, the method further comprising receiving the adjustable shunt in one or both of the first shunt-receiving slot and the second shunt-receiving slot.

9. The method of claim 7 wherein the first tip includes a first shunt-receiving channel and the second tip includes a second shunt-receiving channel, the method further comprising receiving the adjustable shunt within one or both of the first shunt-receiving channel and the second shunt-receiving channel.

10. The method of claim 9 wherein receiving the adjustable shunt within one or both of the first shunt-receiving channel and the second shunt-receiving channel includes receiving a proximal portion of the adjustable shunt between the first tip and the second tip such that a distal portion of the adjustable shunt extends distally beyond one or both of the first shunt-receiving channel and the second shunt-receiving channel.

11. The method of claim 7 wherein positioning the adjustable shunt at the target location further comprises actuating a locking mechanism of the adjustable shunt to allow movement of one or both of the first arm and the second arm relative to one another during operation.

12. The method of claim 11 wherein actuating the locking mechanism includes moving an actuator of the locking mechanism away from the adjustable shunt.

13. The method of claim 7 wherein positioning the adjustable shunt at the target location includes positioning at least a portion of the adjustable shunt posterior to an iris of an eye of the patient.

14. The method of claim 7 wherein positioning the adjustable shunt at the target location includes positioning at least a portion of the adjustable shunt anterior to an iris of an eye of the patient.

15. The method of claim 7 wherein positioning the adjustable shunt at the target location includes positioning a first portion of the adjustable shunt in a first body region of the patient and positioning a second portion of the adjustable shunt in a second body region of the patient.

16. The method of claim 7, further comprising priming the adjustable shunt with a priming fluid before positioning the adjustable shunt within the patient.

17. The method of claim 16 wherein priming the adjustable shunt includes causing the priming fluid to enter at least a portion of the adjustable shunt.

18. The method of claim 16 wherein the implant delivery system includes a cap defining a chamber configured to contain the adjustable shunt, and wherein priming the adjustable shunt includes injecting the priming fluid into the chamber.

19. The method of claim 16 wherein the implant delivery system includes a cap defining a chamber configured to contain the adjustable shunt, and wherein priming the adjustable shunt includes:
generating a vacuum within the chamber, and
drawing, by the vacuum, the priming fluid into the chamber.

20. The method of claim 19, further comprising removing the cap prior to positioning the adjustable shunt at the target location.

21. The method of claim 16 wherein priming the adjustable shunt includes providing the priming fluid to the chamber through one or both of a first end portion of the adjustable shunt and a second end portion of the adjustable shunt.

22. A delivery system for an adjustable shunt for treating a patient, the delivery system comprising:
a first arm including a first tip;
a second arm including a second tip, wherein the second arm is configured to be movably coupled to the first arm; and
a locking mechanism operably coupled to the first arm and configured to releasably engage the second arm to at least partially prevent movement of one or both of the first arm and the second arm relative to one another,
wherein
the first tip and the second tip are each configured to releasably receive the adjustable shunt when the locking mechanism releasably engages the second arm, and
one or both of the first tip and the second tip include a pair of fingers defining a channel therebetween, wherein the channel is configured to receive at least a portion of the adjustable shunt.

23. The delivery system of claim 22 wherein one or both of the pair of fingers are biased toward one another to at least partially prevent movement of the adjustable shunt relative to the channel.

24. The delivery system of claim 22 wherein the channel defines a notch configured to contact a corresponding wing of the adjustable shunt to at least partially prevent movement of the adjustable shunt relative to the channel.

25. The delivery system of claim 24 wherein the channel is configured to receive a first portion of the adjustable shunt, wherein a second portion of the adjustable shunt is configured to extend distally beyond the channel, wherein the channel defines a notch configured to at least partially prevent proximal movement of the adjustable shunt relative to the channel.

26. The delivery system of claim 22 wherein one or both of the first tip and the second tip include a transverse slot extending through the associated first tip or the second tip and through which at least a portion of the adjustable shunt is visible at least when the adjustable shunt is received at the delivery system.

27. The delivery system of claim 22 wherein the first arm and/or the second arm are configured to be biased in a first direction outwardly from one another, and further wherein the first arm and/or the second arm are configured to be movable in a second direction opposite the first direction and inwardly toward one another.

28. The delivery system of claim 22 wherein the first arm includes a slot, and wherein the locking mechanism includes an actuator slidably disposed within the slot.

29. The delivery system of claim 28 wherein the actuation mechanism is configured such that (i) movement of the actuator in a first direction causes the actuation mechanism to at least partially prevent movement of the first arm and the second arm relative to one another, and (ii) movement of the actuator in a second direction opposite the first direction causes the actuation mechanism to allow movement of one or both of the first arm and the second arm relative to one another.

30. The delivery system of claim 22, further comprising a stopping component positioned between the first arm and the second arm, wherein the stopping component is configured to at least partially prevent movement of one or both of the first arm and the second arm toward one another.

31. The delivery system of claim 30 wherein the stopping component extends perpendicularly from a second inner surface portion of the second arm toward a first inner surface portion of the first arm.

32. The delivery system of claim 30 wherein the stopping component includes a stopping surface configured to contact the first inner surface portion in response to movement of one or both the first arm and the second arm toward one another to at least partially prevent further movement of one or both of the first arm and the second arm toward one another.

33. The delivery system of claim 30 wherein the locking mechanism is configured to releasably engage the stopping component to at least partially prevent movement of one or both of the first arm and the second arm toward one another.

34. The delivery system of claim 33 wherein the stopping component includes a recess, and wherein the locking mechanism includes a shaft configured to be positionable within the recess to at least partially prevent movement of one or both of the first arm and the second arm relative to one another.

35. The delivery system of claim 22, further comprising a cap that defines a chamber configured to receive at least a portion of the first tip, the second tip, and the adjustable shunt therein.

36. The delivery system of claim 35 wherein the cap defines an inlet, and wherein the chamber is configured to receive fluid via the inlet.

37. The delivery system of claim 35 wherein the chamber is configured to contain a vacuum generated therein and receive priming fluid drawn from a priming fluid source via the vacuum.

38. The delivery system of claim 35 wherein the cap is configured to form a fluid-impermeable seal with at least one of the first arm, the first tip, the second arm, and the second tip.
